(19) United States
(12) Patent Application Publication
(10) Pub. No.: US 2005/0171447 A1
(43) Pub. Date: Aug. 4, 2005

(54) Method and device for the automateddetection and differentiation of cardiac rhythm disturbances

(76) Inventor: Hans D Esperer, Magdeburg (DE)

Correspondence Address: BACON & THOMAS, PLLC, 625 SLATERS LANE, FOURTH FLOOR, ALEXANDRIA, VA, 22314, US

(21) Appl. No.: 10/498,513

(22) PCT Filed: Dec. 20, 2002
(86) PCT No.: PCT/EP02/14689

(30) Foreign Application Priority Data
Dec. 21, 2001 (DE) 101 63 348.3

(51) Int. Cl. A61B 5/04; A61N 1/18; A61N 1/20; A61N 1/22

(52) U.S. Cl. 600/515; 607/5

(57) ABSTRACT

The invention relates to a method and a device for the automated detection and differentiation of cardiac rhythm disturbances. According to said method, measured values for a coherent series of a cardiac frequency signal, preferably the times of the peaks of the R waves are correlated together using a Poincaré model and the correlated data transformed into a multi-dimensional phase space and represented as a Lorenz curve. The data transformed into the phase space is morphologically characterised and assigned to particular pre-determined patterns by means of case-based reasoning. Particular clinically-relevant rhythmic or arrhythmic forms can be determined from the assigned patterns by means of empirically-determined relationships.

The present invention relates to a method and to a device for the automated detection and differentiation of cardiac rhythm disturbances.

The human heart is a highly developed and complex electromechanical system essential to life. The heart, which typically beats between 100,000 and 140,000 times during a 24-hour period, is admittedly not under voluntary human control, but is very closely linked, via the nerves of the sympathetic and parasympathetic nervous system, to all the organ systems which are important for regulating the circulation, so that cardiac output can be continuously adapted to the constantly changing needs of the circulatory system. When necessary, the heart can increase or decrease the force of its beat and also its beat rate within a few milliseconds. Because of the complex influences of the central nervous system, respiration, circulation and thermoregulation, the heart beat does not represent a constant parameter but instead exhibits, even in healthy humans, an irregular, almost “chaotic” rhythm, which is designated as heart rate variability (HRV).

Recent scientific studies have shown that the cardiac autonomic nervous system is directly related to the development of cardiac rhythm disturbances from the region of the atria (supraventricular arrhythmias) and the ventricles (ventricular arrhythmias). For this reason, studies of heart rate variability have assumed increasing importance in cardiology. To date, however, studies of heart rate variability have been restricted to detection of what is called the autonomic status, or to assessment of the risk of cardiac arrhythmia. However, studies of heart rate variability have not as yet been used in clinical diagnosis and differential diagnosis.

Particularly in connection with so-called event monitoring and telemedical monitoring of cardiac rhythm, also called home monitoring, increasing importance has in recent years been attached also to continuous electrocardiographic monitoring of patients at high risk of spontaneous supraventricular and ventricular arrhythmias.

At present, arrhythmia is monitored mainly by noninvasive techiques, although invasive techniques are used for certain indications, for example syncope of unknown origin.

For patients being treated on an outpatient basis, the noninvasive electrocardiographic (ECG) monitoring methods presently available include the traditional 24-hour Holter monitoring, and also event monitoring and telemetric monitoring. Modern event recorders permit remote transmission of short segments of an electrocardiogram (ECG), of 30 seconds to 5 minutes duration, by telephone modem or mobile telephone; modern event recorders can already be integrated into mobile telephones.

Arrhythmia detection in the Holter analysis devices is based on different methods, above all what are called “Template Matching” and “Feature Extraction” (Kennedy et al., Am J Noninvasive Cardiol 1992; 6: 137-146) and the QRS surface method (Neilson et al., Computers in Cardiology, IEEE catalogue 1974; 74: 379). In recent times, neuronal networks have also been employed to evaluate ECG data. Although nonlinear dynamic methods have already been discussed at the theoretical level by Igel et al. in J Cardiovasc Electrophysiol 1997; 8: 388-397, they have not as yet been implemented in any routinely used clinical analysis system. U.S. Pat. No. 6,192,273 (Igel et al.) describes an automated method for classification of cardiac rhythm, said method being intended to permit detection of normal rhythm, monomorphic tachycardia and polymorphic tachycardia. This method measures the cycle length and the regularity of 5-second measurement intervals.

In the case of inpatients, heart rhythm is monitored either by means of a bedside ECG monitor or centrally via an ECG monitoring apparatus in the intensive care unit.

As regards invasive methods, the companies Reveal and Medtronic presently market implantable rhythm monitors intended for detection of bradyarrhythmias. In these, the ECG signal is stored and has to be read out from an external telemetry device and then assessed by an expert. Automatic detection of arrhythmia is not possible.

The arrhythmia monitoring and detection methods known to date have numerous disadvantages.

Thus, an important disadvantage of the known long-term ECG analysis systems lies in the fact that an expert carrying out the assessment first has to teach the analysis systems the basic rhythm. In addition, the investigating physician normally has to validate the arrhythmia forms initially detected by the analysis system. Finally, the spectrum of the arrhythmia forms which can be detected and differentiated is very small in view of the wide varierty of clinically relevant cardiac rhythm disturbances and typically comprises the following types of rhythm and arrhythmia:
Sinus rhythm
Supraventricular extrasystole (SVES)
Ventricular extrasystole (VES) or aberrant ORS complex
Supraventricular tachycardia (SVT)
Ventricular tachycardia (VT)
Pauses
VVI pacemaker activity (i.e. activity of a permanently implanted single-chamber demand pacemaker)
DDD pacemaker activity (activity of a dual-chamber pacemaker which can operate in different modes (such as WD, VAT or DDD)).
   (The abbreviations WI, WD, VAT and DDD correspond to an internationally recognized pacemaker code).

Whereas technological innovations and an associated improvement in signal quality have greatly enhanced the diagnostic efficiency of detection of ventricular arrhythmias, the detection and differentiation of supraventricular arrhythmias still represents an unsolved problem. Because of the sparsity of the potentials which can be picked up on the skin surface and which represent atrial electrical activity, and because of the high noise levels caused by muscle potentials and movement artefacts, atrial and supraventricular arrhythmias have hitherto completely eluded detection and differentiation. In the presence of a normal heart rhythm (i.e. a sinus rhythm), it is possible to detect and measure the P waves by means of complicated amplification and averaging methods. However, these methods are possible only at rest and fail as soon as the patient resumes normal activity, with the result that they are unsuitable, for example, for outpatient monitoring methods. In addition, the hitherto known methods always analyze an averaged sinus rhythm, so that it is not possible to perform beat-by-beat diagnosis. Moreover, these averaging methods are suitable only when P waves are present, i.e. they cannot be used for diagnosis of atrial arrhythmias which are characterized by the absence of P waves and which make up the majority of atrial arrhythmias.

Likewise, the systems used hitherto for event monitoring do not permit automatic arrhythmia detection, let alone arrhythmia differentiation, because, as before, the arrhythmia analysis has to be checked by an expert.

The telemetry systems which are used in hospitals, and which permit detection of ventricular arrhythmias (for example isolated VES, repetitive VES or ventricular tachycardia) and pauses, are also not suitable in practice for detection and differentiation of supraventricular arrhythmias. Moreover, these systems represent stationary evaluation devices to which the ECG signal has to be sent via portable ECG transmitters. Because of the limited power of the transmitters, the patients always have to remain in the vicinity of the central unit, with the result that outpatient monitoring of heart rhythm under realistic conditions of daily life is not possible.

A more realistic form of patient monitoring is permitted by ECG telemetry via mobile telephone. However, the narrow bandwidth of the mobile telephone systems drastically limits the length of the ECG sections that can be transmitted in this way.

The present invention is therefore based on the technical problem of making available a method and a device which are used for the detection and differentiation of cardiac rhythm disturbances and which permit fully automatic detection of clinically relevant arrhythmias that no longer have to be validated by experts. The method according to the invention is intended to be able to be used both directly at the patient's bedside and also via a central monitoring unit, for example a telemetry station, or also in telematic mode for remote patient monitoring, for example in what is called home monitoring. In addition, the method according to the invention is intended to be able to be used in implantable devices such as pacemakers and defibrillators.

This technical problem is solved by the method for the automated detection and differentiation of cardiac rhythm disturbances in accordance with attached claim 1 and by the corresponding device in accordance with attached claim 9. Advantageous developments of the method according to the invention and of the device according to the invention form the subject of the dependent claims.

The subject of the present invention is therefore, in the first instance, a method for the automated detection and differentiation of cardiac rhythm disturbances, in which method measured values of a coherent time series of a heart rate signal are correlated with one another, and the correlated data are transfomed into a multidimensional phase space, the data transformed into the phase space are morphologically characterized and assigned to certain predetermined patterns, and, finally, certain clinically relevant rhythmic or arrhythmic forms are determined from the assigned patterns on the basis of empirically established relationships.

Accordingly, the present invention essentially proposes a method for nonlinear analysis of heart rate variability in which the original measured values of a time series, namely of the successive heart cycles, are represented in a phase space. The invention is based on the recognition that, in the phase space representation, patterns can be identified which can be assigned with a high level of sensitivity and specificity to certain clinically relevant rhythmic or arrhythmic forms. They are assigned in this case on the basis of empirically established relationships, i.e. on the basis of data from clinical studies which determined and classified the phase space patterns of patients whose rhythmic or arrhythmic forms are known.

The method according to the invention permits fully automatic detection and differentiation of a broad spectrum of clinically relevant supraventricular and ventricular tachyarrhythmias and bradyarrhythmias.

The correlation of the measured values of the time series and the transformation of the correlated data into a multidimensional phase space is preferably done by means of nonlinear methods, for example by means of a chart which, in mathematical terminology, is referred to as a “return map” and has hitherto been used in particular for physical analysis of dynamic processes. The measured values of the coherent time series of the heart rate signal are particularly preferably correlated using what is called a Poincaré model, and the correlated data are represented in what is called a Lorenz curve. A Lorenz curve, which in the literature is also known as a Poincaré curve or scattergram, is a graphic representation in which the interval length of a heart cycle is plotted against one or more previous heart cycle intervals. A general two-dimensional (2D) Lorenz curve is one in which the interval length Tⁿ of the n-th heart cycle is plotted against the interval length T^{n-m}, where m is a constant integer delay. Where m=1, i.e. a heart cycle interval plotted against the immediately preceding interval, this is referred to as a regular 2D Lorenz curve. The resulting local point density of a two-dimensional Lorenz curve can be depicted graphically, for example, by different shades of gray or by a defined color representation. It is also possible to represent the data points of the 2D Lorenz curve in the form of a so-called 3D Lorenz diagram, where, above the (Tⁿ, T^{m-m}) plane, the number of points i is plotted which are situated within a surface unit with edge length τ of the 2D Lorenz curve. The 3D diagram therefore permits better visualization of the point density of the 2D Lorenz curve.

In the literature, three-dimensional Lorenz curves are also known in which the phase space is stretched for example by a Poincaré model of three heart rate intervals (Tⁿ, Tⁿ⁻¹, Tⁿ⁻²).

In a standard work by Woo et al., Am Heart J 1992; 123: 704-710, the time profile of the RR intervals from a 24-h Holter ECG was visualized by means of Lorenz curves. It was possible to differentiate only between healthy subjects and individuals with heart disease, and Woo was able to use the pattern of heart rate variability to draw conclusions regarding the severity of the cardiac insufficiency. However, arrhythmias cannot be determined by the method used by Woo et al. Applications of Lorenz curves to study heart rate variability have also been described by Kamen and Tonkin in Aus NZ J Med 1995; 25: 18-16. However, Kamen and Tonkin do not mention any possibility of using Lorenz curves for the automatic detection and differentiation of arrhythmias. A more recent work by Azuaje et al., Artificial Intelligence in Medicine 1999; 15: 275-297, makes clear the limits of the method itself for determining heart rate variability. This recent work too did not consider detection of arrhythmias. In a work by Huikuri et al., Circulation 1996; 93: 1836-1844, the heart rate variability of patients with known arrhythmia is visualized by means of Lorenz curves. This work investigated in particular the question of whether the sympathetic or the vagus lies at the cause of the development of tachyarrhythmias. These studies too do not indicate the possibility of diagnosis of arrhythmias.

By contrast, the method according to the invention is based on the observation that a high-definition Lorenz curve contains a large amount of data which can be characterized as patterns that can be quantitatively described in terms of geometry and topology, the patterns being able to be assigned to certain dinically relevant rhythmic or arrhythmic forms on the basis of previous empirical investigations. Representation as a 2D Lorenz curve has proven particularly advantageous, although frequency distributions in the sense of a 3D Lorenz diagram are also advantageously taken into consideration.

Automatic, computer-aided morphological characterization of a Lorenz curve can be performed by means of a wide variety of methods, as are known from the field of pattern recognition. For example, neuronal networks can be trained to correlate the Lorenz curves, determined from a patient, with certain predetermined Lorenz curves which are stored in a databank and in which the allocation to certain rhythmic or arrhythmic forms is known. Depending on the amount of data of a 2D Lorenz curve or a 3D Lorenz diagram, the data can also be reworked before pattern allocation, for example smoothed by averaging within a defined surface area.

However, so-called cluster analysis methods have proven particularly advantageous for morphological characterization of Lorenz curves. In these methods, the Lorenz curve is systematically rasterized in a manner known per se, starting from predetermined start positions, and all points lying within a defined radius of the start positions are grouped together to form a minicluster. Moreover, the mean number of point neighbors is calculated. If this number is below a defined limit value, which is referred to as neighborhood threshold, a cluster edge is defined; otherwise, the procedure is continued until the neighborhood threshold is reached. In this way, larger clusters are formed which are demarcated on all sides. The distance distribution of the clusters thus detected is determined and, if it is below a defined limit value, contiguous clusters are fused to form a large cluster. This limit value, which ultimately constitutes the interrupt criterion for cluster formation, is defined empirically in such a way that the resulting cluster patterns are simplified to the extent that a reasonable correlation with empirically established rhythmic or arrhythmic forms is guaranteed. If the limit value chosen is too great, each Lorenz curve in the end shows a single, basically undifferentiated large cluster, which no longer permits diagnostic differentiation. By contrast, if the limit value chosen is too small, this results in a large number of different patterns whose morphological differences no longer correspond to any suitably differentiable arrhythmia forms. A suitable method for cluster formation is described, for example, in Lanzarini et al., First Int'l Workshop on Image and Signal Processing and Analysis, 14.-15 Jun. 2000, Pula, Croatia, pages 75-80.

The patterns determined are geometrically parameterized in respect of edge and midpoint coordinates, for example in the form of a vector matrix with polar coordinates. The position vectors of the cluster edge can be divided, in terms of coordinate origin, into distal and proximal position vectors. The local maxima of the individual clusters are also analyzed. The geometric shape criteria and frequency measurements thus determined are assigned to certain pattern classes via a decision matrix. These are once again preferably assigned using neuronal networks, using fuzzy logic methods, for example fuzzy clustering and/or fuzzy control, or, particularly preferably, using methods of artificial intelligence, for example case-based reasoning (CBR method). The predetermined patterns are now once again correlated in a databank with certain clinically relevant rhythmic or arrhythmic forms, so that, after allocation of a measured pattern to a predetermined pattern with the aid of the databank, corresponding detection and differentiation of cardiac rhythm disturbances is possible.

The measured values of the coherent time series can be collected and evaluated over any desired lengths of time. For example, it is conceivable to represent the measured values of a 24-hour measurement in a single Lorenz curve. However, such a representation provides an excess of information, and there is in particular a danger that individual, clinically relevant data items will no longer be able to be detected properly. It is therefore preferable for the collected data to be segmented into shorter time series, and in this case it is particularly preferable for the measured values of the coherent time series of the heart rate signal to cover a period of 1 to 15 minutes, preferably 1 to 10 minutes, and particularly preferably a period of about 5 minutes. A first practicable allocation of a heart rate measurement to a certain pattern can typically take place after ca. 30 heart cycles, that is to say after a measurement duration of ca. 1 minute. In certain cases however, for example in continuous patient monitoring, certain clinically relevant early indicators of arrhythmia may also be registered with a substantially shorter time resolution. Thus, it is known that certain life-threatening arrhythmias, for example tachycardias, are often heralded by a reduction in heart rate variability (so-called “heart rate stiffness”). In continuous monitoring, an early indicator, like this, of imminent arrhythmia may often also be detected after less than 30 heart cycles.

According to a particularly preferred embodiment of the invention, the databank with the empirically established relationships between patterns and rhythmic or arrhythmic forms contains both a long-term database (for example on the basis of 24-hour data) and also a short-term database (for example on the basis of 5-minute segments).

Determination of the clinically relevant rhythmic or arrhythmic forms preferably takes into consideration not just the morphological data of pure pattern recognition, but also the quantification of heart rate variability. This can take place in the phase space or in the time range, but preferably both in the phase space and also in the time range.

It is possible to use, as the heart rate signal, a wide variety of measurement parameters which permit continuous monitoring of the heart rate, for example the pulse curve or the blood pressure curve. However, it is particularly preferable to use a digitized ECG signal which can be evaluated in the time range using methods known per se. The ECG can in this case be recorded as an analog signal directly from the patient, for example via the corrected orthogonal leads x, y and z as per Frank. The ECG leads undergo analog-digital conversion, preferably after bandpass filtering and amplification. The resolution is advantageously in the range of 8 to 24 bit, and the scanning frequency is advantageously 100 Hz to 1 kHz.

It is also possible to use, as input signal, ECG signals which have already been digitized and which are transmitted by telemetry, for example via the Internet or a digital telephone or data link, to an analysis unit. In principle, the data can also be evaluated on the basis of a one-channel ECG.

To calculate the time duration of a heart cycle, the interval between two successive R waves of the ECG signal is advantageously determined. The peak of the R wave can be determined reliably and with great accuracy using what are known as QRS detection algorithms, which are known per se. This measurement provides, relative to the resolution of the digitization, integer values which are stored as a so-called RR tachogram. The RR tachogram data from long-term monitoring are segmented into short-term blocks. For example, from a 24-hour ECG monitoring, it is possible to obtain 228 blocks of 5 minutes each, which are then processed to give 288 Lorenz curves and are analyzed.

The method according to the invention can be implemented in particular using software technology, with access to a databank in which the empirical clinical data are stored. The present invention therefore also relates to a computer program on a computer-readable carrier, which comprises program instructions for performing the method according to the invention on a computer. The computer-readable carrier can, for example, be a storage medium such as a CD-ROM or DVD-ROM, a hard disk, a diskette, or even a storage element of a computer, for example ROM or EPROM storage elements. A computer-readable carrier in the sense of the present invention also includes electrical carrier signals, such as are used, for example, in the Internet when downloading programs from an external server to a local computer.

The present invention also relates to a device for the automated detection and differentiation of cardiac rhythm disturbances, with an input unit which detects heart rate signals, a processing unit which evaluates the detected heart rate signals and determines associated rhythmic or arrhythmic forms, and an output unit which outputs the determined rhythmic or arrhythmic forms. The processing unit of the device according to the invention is essentially a computer which comprises storage and processor elements and in which a computer program as mentioned above is implemented, essentially comprising the following preferably software-based means:
means for determining the duration of successive heart beat cycles of the heart rate signal;
means for correlating each cycle duration with one of the preceding cycle durations, preferably with the immediately preceding cycle duration, and for transforming the correlated data into at least one Lorenz curve;
means for morphological characterization of the Lorenz curve and for assigning the Lorenz curve to predetermined patterns;
and databank means which correlate the predetermined patterns with certain clinically relevant rhythmic or arrhythmic forms.

The device according to the invention can be configured, for example, as a portable device and can be implemented, for instance, in a notebook which has interfaces known per se for input and output of data.

According to one embodiment, the device according to the invention is simply an evaluation unit, in which case the input unit comprises means for reading-in, particularly for telemetric reception of optionally digitized heart rate signals. If the heart rate signals are to be read-in in analog form, the input unit also comprises means for digitizing the heart rate signal.

According to another embodiment, the input unit comprises suitable means for recording and digitizing heart rate signals, preferably ECG signals, which can be realized, for example, in the form of electrode leads known per se. The input unit can also have means for continuous recording of blood pressure or means for recording the pulse, from which, in turn, the interval durations of the heart rate cycles can be determined. The output unit of the device according to the invention can have means for displaying the determined rhythmic or arrhythmic forms and/or suitable alarm means for indicating life-threatening arrhythmias. Means are preferably provided for telemetric relay of the determined rhythmic or arrhythmic forms to a medical practice or hospital. The data transfer can take place, for example within a hospital, via a local area network (LAN) or, if longer distances are involved, by telephone or Internet. If pathological arrhythmias are detected, it is possible to transmit, in addition to the automatically detected arrhythmia form, also the associated heart rate signals in the time range, so that the physician in attendance is provided with further information for the decision to instigate the necessary measures.

The present invention has a wide diagnostic potential and permits, for example, the fully automatic detection and differentiation of the following forms of heart rhythm and arrhythmia:
Sinus rhythm with normal HRV
Sinus rhythm with reduced HRV
Sinus rhythm with severely reduced HRV
VES with compensatory pause and fixed coupling interval
VES with variable coupling interval (parasystole)
VES bigeminy
Polytope VES
NSVT (nonsustained ventricular tachycardia)
VT (sustained ventricular tachycardia)
SVES (supraventricular extrasystole)
SVES bigeminy
Persistent AF (atrial fibrillation)
Paroxysmal AF (atrial fibrillation)
Atrial flutter (AFL) with alternate AV conduction
Atrial tachycardia (AT) with alternate AV conduction
Bradycardia-associated pauses
VES-associated pauses
Artefact-associated pauses
Artefacts/tape errors

The device according to the invention is suitable in particular for monitoring centers where a large number of at-risk patients have to be carefully monitored in real time. Here, the routine continuous monitoring can be carried out fully automatically. Closer monitoring, if appropriate by a physician, is only needed, for example, when the method according to the invention and/or the device according to the invention has/have detected certain pathological arrhythmia forms which require initiation of precautionary or acute treatment measures.

The method according to the invention and the device according to the invention can also be integrated into implantable defibrillators or pacemakers. The devices thus equipped can, for example, give early warning of the danger of life-threatening tachycardia by detecting a reduction in heart rate variability. In such a case, the capacitors of the defibrillator can be charged as a precautionary measure, again automatically, so that a shock can be delivered immediately after onset of tachycardia and not, as in known defibrillators, after a delay period of up to one minute associated with the charging of the capacitors. The present invention therefore also relates to an implantable defibrillator with a defibrillation arrangement, a voltage source and an impulse transmitter which has a chargeable capacitor, and a device according to the invention for the detection and differentiation of cardiac rhythm disturbances is integrated into the defibrillator.

The method according to the invention and the device according to the invention are extremely robust against disturbances, for example wandering of the ECG baseline and interference voltages caused by muscle artefacts. In the context of the differentiation of supraventricular arrhythmias, this proves to be a great advantage since, with the present invention, low-amplitude ECG waves, for example P waves, are not needed for the detection and differentiation of arrhythmias.

Since, according to a preferred embodiment of the method according to the invention, detection of the QRS peaks of the ECG suffices alone, the method according to the invention is particularly suitable for monitoring arrhythmia under conditions of everyday life, for example at the workplace or when taking part in sports activities.

The method according to the invention leads to a considerable improvement in the reliability and accuracy of long-term and short-term analysis of heart rhythm. In particular, the combination of the method according to the invention with conventional long-term ECG and arrhythmia monitoring leads to a considerable improvement in the quality of the findings.

The method according to the invention also considerably improves the capacity of long-term monitoring facilities, for example so-called Holter laboratories, because, on the one hand, fewer members of staff are needed for evaluation, and, on the other hand, an extension of the monitoring capacity is made possible. Particularly in the continuous operation of a telemedical 24-hour arrhythmia monitoring and evaluation service, the number of pathological and serious arrhythmia findings which require rapid or immediate treatment will increase as a function of the number of patients being monitored simultaneously. Therefore, automatic preselection of normal and pathological findings is an important precondition for reliable and economically feasible simultaneous monitoring of a large number of patients.

The invention is explained in more detail below on the basis of a preferred illustrative embodiment in which the heart rate signals are in the form of ECG signals. Reference is made to the attached drawings, in which:

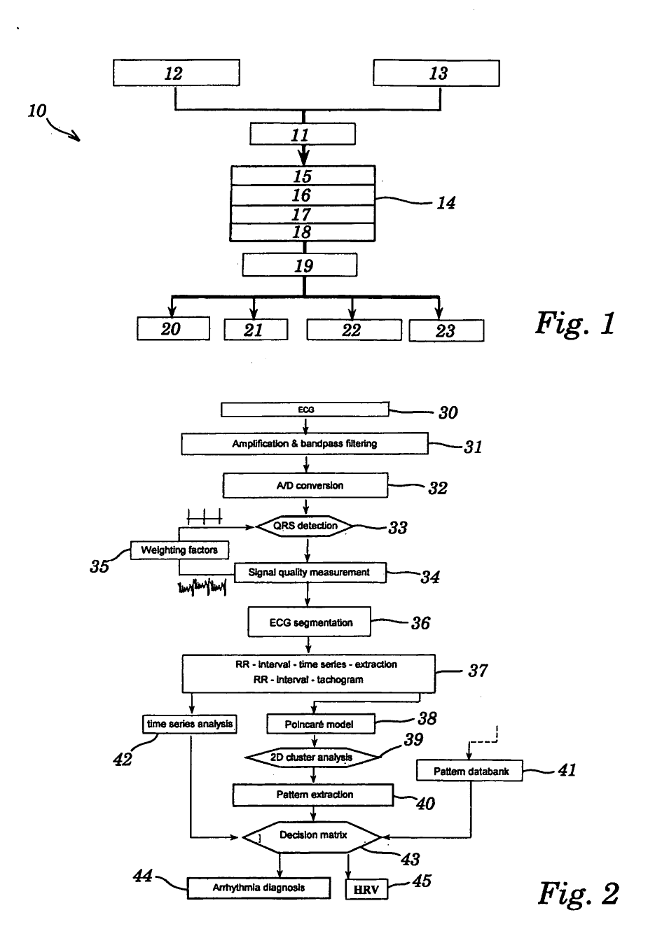

FIG. 1 shows the basic structure of a preferred embodiment of the device according to the invention for the automated detection and differentiation of cardiac rhythm disturbances;

FIG. 2 shows a flowchart illustrating how the method according to the invention is carried out;

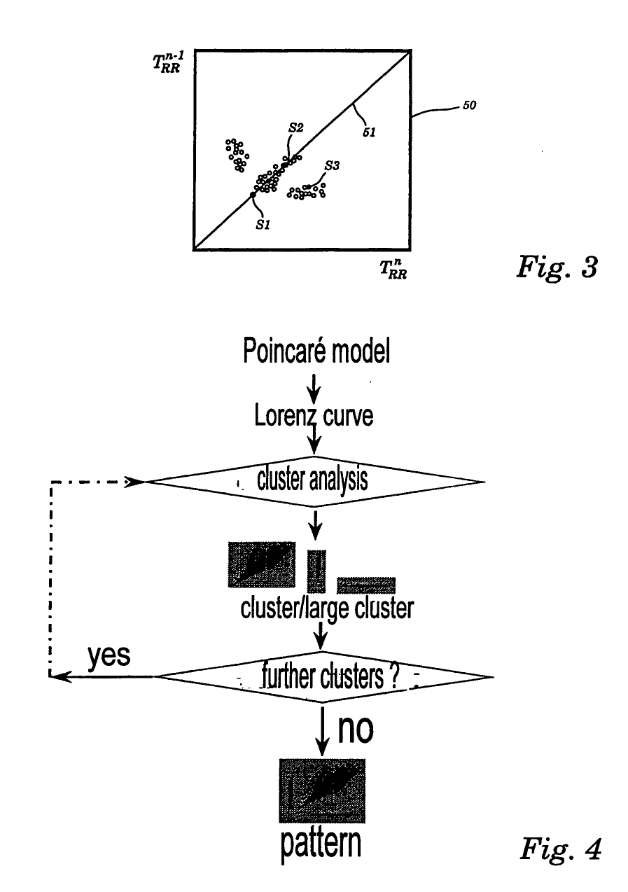

FIG. 3 shows a diagrammatic Lorenz curve;

FIG. 4 shows a diagrammatic representation of the automatic detection of patterns in a Lorenz curve by means of cluster analysis;

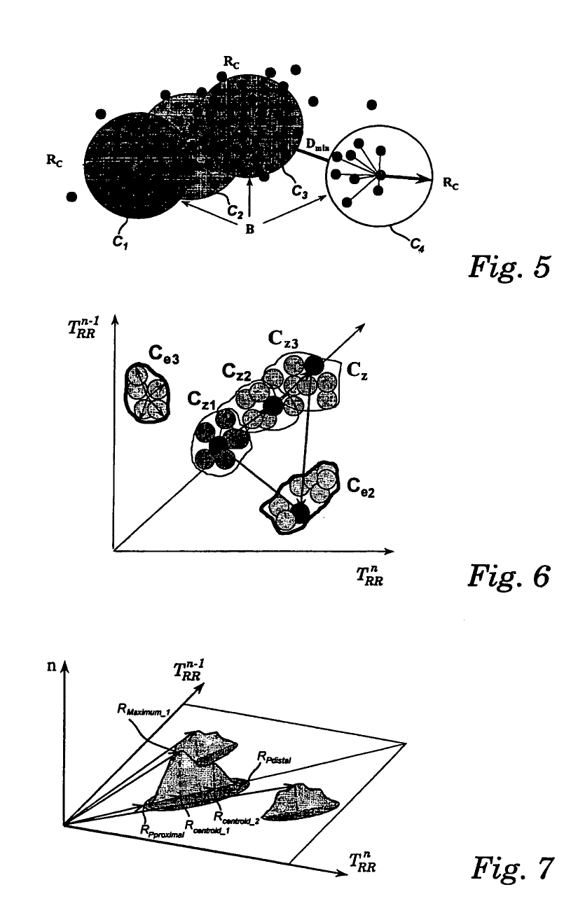

FIG. 5 shows an illustration of cluster fusion in pattern recognition in the Lorenz curve;

FIG. 6 shows a Lorenz curve with three detected large clusters;

FIG. 7 shows an illustration of important parameters for morphological characterization of the large clusters which have been determined;

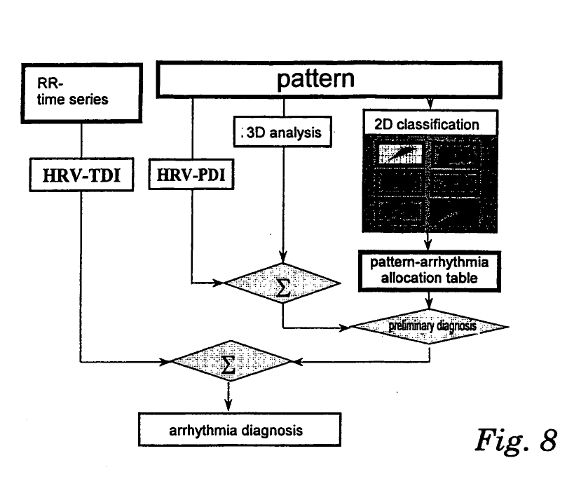

FIG. 8 shows a diagrammatic representation of the automated detection of arrhythmia forms from pattern data and time series analyses.

FIG. 1 is a diagram showing the structure of a device 10 according to the invention for the automated detection and differentiation of cardiac rhythm disturbances. The device according to the invention comprises an input unit 11 which detects heart rate signals, the heart rate signals being delivered via a single-channel ECG 12 or a multi-channel ECG 13. The device according to the invention also includes a processing unit 14 which evaluates the detected heart rate signals and determines associated rhythmic or arrhythmic forms. The processing unit 14 can have means for ECG preparation 15, for example amplifier and/or filter, an analog/digital converter 16, and other signal processing means. The core elements of the processing unit 14 are means for arrhythmia analysis 17 and means for heart rate variability analysis 18, which means will be discussed in more detail below. The device 10 also has an output unit 19 which can output the determined rhythmic or arrhythmic forms in different ways, for example on a monitor 20, a printer 21, a CD burner 22, or a telemetry unit 23, which can also transmit the data by modem via telephone or data links, for example the Internet.

The processing unit of the device according to the invention can be realized, for example, in the form of a notebook equipped with special analysis software. The input unit for detection of ECG signals can, for example, have a conventional arrangement of ECG electrodes, cables and distributor box, for example for Frank orthogonal leads. The analog ECG signals are digitized with a scanning frequency of 500 Hz and a resolution of 12 bit and are transmitted via a light wave guide to a serial interface of the notebook provided with an optocoupler.

The method according to the invention for the detection and differentiation of cardiac rhythm disturbances is now discussed in more detail with reference to FIG. 2.

In a first step, designated by reference number 30, an ECG is recorded, for example with orthogonal x, y, z leads, and is then amplified in step 31 and filtered via a bandpass (0.05-500 Hz). In step 32, analog/digital conversion takes place at 12 bit and 500 Hz scanning frequency. A QRS detection algorithm 33 determines the peak of the R wave of the digitized heart cycles. If appropriate, in a step 34, the signal quality can be determined for each heart cycle, and the recorded R waves can be correspondingly provided with weighting factors 35. In step 36, the ECG cycles are segmented into 5-minute blocks, from which, in step 37, RR interval series are then extracted which, in the example shown, are also designated as RR tachograms. The 5-minute tachograms are analyzed in two different ways: on the one hand, a Poincaré model 38 is used for transformation into a two-dimensional phase space, which is designated as a Lorenz curve. By means of a two-dimensional cluster analysis 39, the measured Lorenz curve is assigned in step 50 to certain predetermined patterns. These patterns are compared to an empirically established databank 41 in which a correlation of the predetermined patterns with certain rhythmic or arrhythmic forms is stored. On the other hand, for fine diagnosis, certain parameters of heart rate variability are determined in a time series analysis 42 from the RR tachograms 37 and are linked in a decision matrix 43 with the results of the morphological evaluation. These data are used as a basis for automatic arrhythmia diagnosis 44 and assessment of heart rate variability (HRV) 45.

A Lorenz curve 50 determined from an RR tachogram is shown diagrammatically in FIG. 3. An RR tachogram contains the interval durations of successive heart cycles, to be more exact the interval duration between successive peaks of the R wave of the heart cycle. In the Lorenz curve, the duration of each interval Tⁿ_{RR} is plotted against the duration of the immediately preceding interval Tⁿ⁻¹_{RR} (except for the first interval, of course, which has no “immediately preceding” interval). Each pair of successive intervals accordingly corresponds to a point of the Lorenz curve. The axis lengths of the Lorenz curve are advantageously chosen, according to generally accepted heart rate criteria, at from 0 to 2000 ms, so that a distinction can be made between tachycardia (0 to 599 ms), a normal rate (600-1199 ms) and bradycardia (1200-2000 ms). On the main diagonal 51, the successive intervals are of the same length. It has now been found that such a representation of successive heart cycle durations yields characteristic patterns of point clouds which closely correlate with certain arrhythmia forms. Such a correlation can be established in the form of a databank after clinical studies of a very large patient population.

For automatic analysis of cardiac rhythm disturbances, it is now necessary to automatically identify the predetermined characteristic patterns in a measured Lorenz curve, i.e. a Lorenz curve obtained from ECG data. In the example shown here of the method according to the invention, pattern recognition is carried out with the aid of so-called cluster analysis. The Lorenz curve 50 is systematically rasterized starting from three predetermined start positions S1, S2 and S3, of which advantageously at least one lies on the main diagonal 51. All points lying within a defined radius are grouped together to form a minicluster. The mean number of point neighbors is also calculated. If this number is below a defined limit value referred to as a neighborhood threshold, a cluster edge is defined. Otherwise, the procedure is continued until the neighborhood threshold is reached. In this way, larger clusters are formed which are demarcated on all sides. In the next step, the distance distribution of the detected clusters is determined. If the distance between two clusters is below a defined limit value D_{θ}, the two clusters are fused to form a large cluster. In heart rate analysis, a suitable value D_{θ} is in the range of 80 to 120 ms, advantageously ca. 100 ms.

The cluster analysis procedure is summarized diagrammatically in FIG. 4. Individual clusters are first determined until the whole Lorenz curve is rasterized. Combination of all the clusters determined in a Lorenz curve yields the sought-after pattern, which is then morphologically characterized.

FIG. 5 shows the procedure involved in cluster fusion. The clusters C1 and C3 are at a distance from one another smaller than the limit value D_{θ}=100 ms, whereas the clusters C3 and C4 are at a distance Dₘᵢₙ of more than 100 ms. Accordingly, the clusters C1 and C3 are fused to form a large cluster, the cluster edge in area C2 accordingly being adapted for determination of the neighborhood threshold. C4 is not fused with the large cluster thus formed. The result of a typical cluster analysis of an entire Lorenz curve is shown in FIG. 6. In the example shown, a central long cluster C_{Z} was obtained, consisting of three fused clusters C_{Z1}, C_{Z2} and C_{Z3}, and two eccentric clusters Cₑ₂ and Cₑ₃.

The clusters or large clusters found by cluster analysis can be qualitatively and quantitatively characterized in many different ways. In the text below, a classification plan recognized as being especially advantageous for characterizing the Lorenz curve is explained in more detail with reference to the diagram in FIG. 7.

As has also already been mentioned in connection with FIG. 6, the clusters found are initially characterized, in terms of their position on or beside the main diagonal of the Lorenz curve, as being “central” or “eccentric” clusters. In addition, the clusters determined are parameterized geometrically in respect of their edge coordinates and midpoint coordinates. As is shown in the 3D Lorenz diagram in FIG. 7, the local frequency maxima of the RR interval distribution within the clusters are also determined. The points located closer to the coordinate origin are referred to as “proximal” and the more distant points are referred to as “distal”. In the following tables, a corresponding set of parameters is shown which is particularly suitable for characterizing a Lorenz curve pattern:

TABLE 1
Parameters for characterizing central clusters

| Form Index | | Definition |
|---|---|---|
| Distal edge vector | R_{Pdistal} | Vector of 0.0 to the distal cluster |
| | | edge |
| Proximal edge vector | R_{Pproximal} | Vector of 0.0 to the proximal |
| | | cluster edge |
| Centroid vector 1 | R_{centroid}_{_{—}}₁ | Vector of 0.0 to the prox. |
| | | maximum base point |
| Centroid vector 2 | R_{centroid}_{_{—}}₂ | Vector of 0.0 to the distal |
| | | maximum base point |
| Local maximum vector 1 | R_{Maximum}_{_{—}}₁ | Vector of 0.0 to the proximal |
| | | maximum |
| Local maximum vector 2 | R_{Maximum}_{_{—}}₂ | Vector of 0.0 to the distal |
| | | maximum |

TABLE 2
Parameters for characterizing proximal eccentric clusters
(Proximal maximum vectors)

| Form Index | Definition |
|---|---|
| Local maximum vector left 1 | rLM1_p |
| Local maximum vector left 2 | rLM2_p |
| Local maximum vector left 3 | rLM3_p |
| Local maximum vector right 1 | rRM1_p |
| Local maximum vector right 2 | rRM2_p |
| Local maximum vector right 3 | rRM3_p |

TABLE 3
Parameters for characterizing distal eccentric clusters
(Distal maximum vectors)

| Form Index | Definition |
|---|---|
| Local maximum vector left 1 | rLM1_d |
| Local maximum vector left 2 | rLM2_d |
| Local maximum vector left 3 | rLM3_d |
| Local maximum vector right 1 | rRM1_d |
| Local maximum vector right 2 | rRM2_d |
| Local maximum vector right 3 | rRM3_d |

In addition to morphological criteria, the heart rate variability (HRV) is also characterized with the aid of conventional time range indices according to Table 4 below, but also by means of phase space indices according to Table 5 following.

TABLE 4
HRV indices from time range

| Index | Unit | Definition |
|---|---|---|
| RRₘᵢₙ | ms | minimum RR interval |
| RRₘₐₓ, | ms | maximum RR interval |
| RRₘₑₐₙ, | ms | arithmetic mean of the RR interval distribution |
| RR_{mode} | ms | mode of the RR interval distribution |
| RR_{median} | ms | median of the RR interval distribution |
| SDNN | ms | standard deviation of the mean RR interval distribution |
| NN50 | 1 | absolute incidence of the differences of successive RR |
| | | intervals greater than 50 ms |
| rMSSD | ms | mean quadratic difference of successive RR intervals |
| ΔNNₘₑₐₙ, | ms | mean difference of successive RR intervals |

TABLE 5
HRV indices from phase range

| Index | Definition |
|---|---|
| Lₘₐₓ | maximum length of the central point cloud |
| Bₘₐₓ | maximum width of the central point cloud |
| B₁₀₀₀ | central point cloud width at 1000 ms |
| B₅₀₀ | central point cloud width at 500 ms |

Despite the immense diversity of the details of the individual Lorenz curve patterns, it was found that certain striking morphological features can be observed particularly frequently. It was further found that certain characteristic Lorenz curve patterns correlate with certain clinically relevant rhythmic or arrhythmic forms.

These characteristic patterns can be divided morphologically, for example as shown in Table 6 below, into subcategories, and these in turn into subordinate prototype classes. The names given to the classes are based on the visual appearance of the respective patterns:

The monomorphic patterns 1-6 consist of a single central cluster, whose shape could be further differentiated with one of the following attributes: conical (=comet), ellipsoid (=torpedo), circular (=discus), fan-shaped (=fan).

The polymorphic patterns 6-10 are composed of several point clouds. Thus, the archipelago type is characterized by central and eccentric point clouds scattered like islands. The bifoliate type is like a petal with two leaflets, while the trifoliate type (three leaflets) is characterized by three lobes. The wing-pair prototype is characterized by the combination of a central, comet-shaped or torpedo-shaped point cloud, and of two strictly axially symmetrically extending contralateral lobes which form an acute angle with the central point cloud. The kite prototype is present in a diamond-shaped or in an arrowtip-shaped morphology, where the smooth right and upper edges of the clusters are particularly apparent. The fragment prototype is characterized by a highly symmetrical distribution of a large number of cluster fragments in the Lorenz curve, and these can be torpedo-shaped (T), fan-shaped (F) or trifoliate (D).

Accordingly, the prototype patterns of categories Ia and Ib can in turn be further subdivided, as is shown in Table 7, by introduction of subtypes. Thus, as has been explained above, the fragment prototype can be subdivided into T, F and D subtypes.

TABLE 6
Pattern categories and classes

| Category | Prototype class |
|---|---|
| Ia simple oligomorphic patterns | 1. comet |
| | 2. torpedo |
| | 3. discus |
| | 4. fan |
| Ib simple polymorphic patterns | 5. archipelago |
| | 6. bifoliate |
| | 7. trifoliate |
| | 8. wing-pair |
| | 9. kite |
| | 10. fragments |
| II combined patterns | combinations of two pattern classes of |
| | category Ia and Ib |
| III complex patterns | combinations of more than two pattern |
| | classes of categories Ia and Ib |

TABLE 7
Subtypes of the prototype patterns

| | |
|---|---|
| Class Ia | |
| Comet | S comet (standard morphology) |
| | E comet |
| | R comet |
| | B comet |
| Torpedo | S torpedo (standard morphology) |
| | L torpedo |
| | F torpedo |
| Discus | — |
| Fan | E fan |
| | D fan |
| Class Ib | |
| Archipelago | 2-island subtype |
| | 4-island subtype |
| | 9-island subtype |
| Bifoliate | Subtype A |
| | Subtype B |
| | Subtype C (lyre) |
| | Subtype D |
| Trifoliate | Subtype A |
| | Subtype B (rotor) |
| | Subtype C (butterfly) |
| Wing-pair | Subtype A |
| | Subtype B |
| | Subtype C |
| Fragment | T subtype (torpedo-shaped) |
| | F subtype (fan-shaped) |
| | D subtype (trifoliate) |

A detailed description of this classification, and the allocation of the patterns to certain rhythmic or arrhythmic forms based on comprehensive clinical studies, is to be found in the postdoctoral thesis written by the Applicant (cf. Dr Hans Dieter Esperer, postdoctoral thesis, University of Magdeburg, 2001).

These predetermined patterns are now characterized by empirically established parameters in accordance with the sets of parameters described above. The empirically established mean values of these parameters can be described by fuzzy terms. A measured Lorenz curve pattern is then allocated to a predetermined pattern, according to a plan referred to as fuzzy interference, by a large number of decision rules (IF-THEN rules), which can also include the expert knowledge gained empirically through clinical studies.

Pattern recognition is effected particularly preferably by implementing a method from artificial intelligence, namely a CBR technique LCase Based Reasoning). The principles of this method are described for example in Lenz, M., Bartsch-Spörl, B., Burkhard, H.-D., Wess, S. (Eds.), “Case-Based Reasoning Technology—From Foundations to Applications”, Lecture Notes in Artificial Intelligence 1400, Springer, 1998 or in Aamodt, A. and Plaza, E., “Case-Based Reasoning: Foundational Issues, Methodological Variations, and System Approaches”, Al Communications, 7 (1): 39-59, 1994. In the context of the present invention, the Lorenz curve pattern to be classified is compared, in terms of several qualitative and quantitative form criteria, to the patterns stored in the database (Lorenz curve pattern lexicon). The pattern having the greatest similarity to the Lorenz curve pattern to be analyzed is identified and used as classification category for the new pattern. The similarity between the pattern to be analyzed and the Lorenz curve patterns present in the database is determined using qualitative and quantitative measurements of similarity. The qualitative measurements of similarity are defined in the present case as yes/no decisions. Examples of these are the existence or absence of one or more central clusters, the existence or absence of paired or unpaired eccentric clusters, etc. Quantitative measurements of similarity are the euclidian differences from geometric and parameterized variables. Geometric variables are, for example, Lₘₐₓ and Bₘₐₓ of the central cluster(s) and Lₘₐₓ and Bₘₐₓ of the eccentric clusters (cf. Table 5). Parameterized variables are, for example, characteristic polar coordinates (cf. Table 1) and the local incidence maxima and minima (cf. Tables 2 and 3).

On the basis of the abovementioned clinical studies, and taking into consideration the data from several thousand patients on whom a 24-hour long-term ECG had been carried out for arrhythmia or suspected arrhythmia, a pattern databank was set up which correlates the above-described patterns and the corresponding clinically determined rhythmic or arrhythmic forms with one another. The 24-hour data and also the 5-minute data sets obtained therefrom were used as database.

As is shown in FIG. 8, the final automatic arrhythmia diagnosis uses not only the morphological data from the 2D and 3D analysis of the Lorenz curve or diagram, but also data on heart rate variability in phase space (HRV-PDI) and in the time range (HRV-TDI). Accordingly, the databank also contains such empirically determined values. Table 8 below gives an outline of a pattern-arrhythmia allocation table which forms the basis for assigning specific arrhythmia forms to the respectively determined patterns.

TABLE 8
pattern - arrhythmia allocation matrix

| | Arrhythmia | |
|---|---|---|
| pattern | main diagnosis | differential diagnosis |
| S comet | Sinus rhythm + normal HRV | DDD pacemaker activity: |
| | | Atrially triggered ventricle |
| | | stimulation with DDD pacing |
| | | and normal chronotropic |
| | | competence |
| E comet | Sinus rhythm + normal HRV | None |
| R comet | Sinus rhythm + normal HRV + intermittently | None |
| | wandering atrial pacemaker | |
| Torpedo | Sinus rhythm + reduced HRV | VVIR pacemaker activity |
| | | rate-adapted single-chamber |
| | | stimulation (VVIR) |
| Discus | Sinus rhythm + severely reduced | 1. SVT: |
| | HRV | 1a AFL with 1:1 cond. |
| | | Ib AVNRT |
| | | Ic AVRT |
| | | 2. VT |
| E fan | Atrial fibrillation | 1. Multifocal tachycardia |
| | | 2. Frequent SVES |
| D fan | Atrial fibrillation | None |
| Archipelago | Atrial flutter with alternate AV | Atrial tachycardia with |
| | conduction | alternate AV conduction |
| Bifoliate | SVES | Interposed VES |
| Trifoliate | | |
| Subtype A | VES | SVES with compensated |
| | | postextrasystolic interval |
| Subtype B | VES with variable coupling | None |
| Subtype C | VES | None |
| Wing-pair | Bradyarrhythmia-associated | 1. VES-associated pauses |
| | pauses | 2. Artefact-associated pauses |
| F kite | VVI activity + atrial fibrillation | none |
| T kite | VVI activity + sinus rhythm | none |
| Fragment | Artefacts/tape errors | none |

The fragment pattern is relevant only in conventional recording.

1-14. (canceled)

15. A method for the automated detection and differentiation of cardiac rhythm disturbances, comprising the steps:
measured values of a coherent time series of a heart rate signal are correlated with one another, and the correlated data are transformed into a multidimensional phase space,
the data transformed into the phase space are morphologically characterized and assigned to certain predetermined patterns,
certain clinically relevant rhythmic or arrhythmic forms are determined from the assigned patterns on the basis of empirically established relationships.

16. The method of claim 15, in which the measured values of the coherent time series of the heart rate signal are correlated using a Poincaré model, and the correlated data are represented as a Lorenz curve.

17. The method of claim 16, in which, for morphological characterization of the Lorenz curve, a cluster analysis is performed which parameterizes detected clusters in the phase space and assigns them to the predetermined patterns on the basis of the determined parameters.

18. The method of claim 15, in which the measured values of the coherent time series of the heart rate signal cover a time period of 1 to 15 min, preferably 2 to 10 min, and particularly preferably about 5 min.

19. The method of claim 15, in which the heart rate variability is also included in the determination of the clinically relevant rhythmic or arrhythmic forms.

20. The method of claim 15, in which a digitized ECG signal is used as the heart rate signal.

21. The method of claim 20, in which the interval between two successive R waves of the ECG signal is in each case used as a measured value for the time series.

22. A computer program on a computer-readable data carrier, which comprises program instructions for performing the method as claimed in claim 15 on a computer.

23. A device for the automated detection and differentiation of cardiac rhythm disturbances, with an input unit which detects heart rate signals, a processing unit which evaluates the detected heart rate signals and determines associated rhythmic or arrhythmic forms, and an output unit which outputs the determined rhythmic or arrhythmic forms, said processing unit comprising:
means for determining the duration of successive heart beat cycles of the heart rate signal,
means for correlating each cycle duration with one of the succeeding cycle durations and for transforming the correlated data into at least one Lorenz curve,
means for morphological characterization of the Lorenz curve and for assigning the Lorenz curve to predetermined patterns,
databank means which correlate the predetermined patterns with certain clinically relevant rhythmic or arrhythmic forms.

24. The device of claim 23, wherein said input unit comprises means for telemetric reception of optionally digitized heart rate signals.

25. The device of claim 23, wherein said input unit comprises means for recording and digitizing heart rate signals, said heart rate signals preferably comprising ECG signals.

26. The device of claim 23, wherein said processing unit is adapted to evaluate at least one coherent time series of the heart rate signal, said time series covering a time period of 1 to 15 min, preferably 2 to 10 min, and particularly preferably about 5 min.

27. The device of claim 23, wherein said output unit comprises means for telemetric relay of the determined rhythmic or arrhythmic forms and, if appropriate, of the associated heart rate signals.

28. An implantable defibrillator with a defibrillation electrode arrangement, a voltage source and an impulse transmitter which has a chargeable capacitor, wherein the defibrillator comprises a device as claimed in claim 23.
